(19) United States
(12) Patent Application Publication
(10) Pub. No.: US 2012/0197402 A1
(43) Pub. Date: Aug. 2, 2012

(54) INTERVERTEBRAL IMPLANT WITH ROTATING MEMBER

(75) Inventors: Jonathan Blackwell, Arlington, TN (US); Anthony Melkent, Memphis, TN (US); Keith Miller, Germantown, TN (US); Kidong Yu, Memphis, TN (US)

(73) Assignee: Warsaw Orthopedic, Inc, Warsaw, IN (US)

(21) Appl. No.: 13/015,192

(22) Filed: Jan. 27, 2011

(51) Int. Cl. A61F 2/44

(52) U.S. Cl. 623/17.16

(57) ABSTRACT

An implant for spacing apart skeletal structures. The implant includes a base, a rotatable member, and a connector. The base extends around at least a portion of the member, and the member is pivotally connected to the base by the connector. The member includes a first pair of opposing sides that have a first height that is smaller than or equal to a height of the base, and a second pair of opposing sides that have a second height larger than the base. The member is movable to a first rotational position with the first pair of opposing sides facing in the same direction as the sides of the base such that the member is shorter than or equal to the base. The member is also movable to a second rotational position with the second pair of opposing sides facing in the same direction as the sides of the base such that the member is taller than the base.

### BACKGROUND

The present application is directed to an implant, and more particularly, to an implant with a non-rotating base and a rotating member that is movable between first and second rotational positions.

Various medical procedures use an implant that is positioned within the patient between supporting skeletal structures. One example is an implant positioned between vertebral members of the spine. The spine is divided into four regions comprising the cervical, thoracic, lumbar, and sacrococcygeal regions. The cervical region includes the top seven vertebral members identified as C1-C7. The thoracic region includes the next twelve vertebral members identified as T1-T12. The lumbar region includes five vertebral members L1-L5. The sacrococcygeal region includes nine fused vertebral members that form the sacrum and the coccyx. The vertebral members of the spine are aligned in a curved configuration that includes a cervical curve, thoracic curve, and lumbosacral curve. Intervertebral discs are positioned between the vertebral members and permit flexion, extension, lateral bending, and rotation.

Various conditions may lead to damage of the intervertebral discs and/or the vertebral members. The damage may result from a variety of causes including but not limited to a specific event such as trauma, a degenerative condition, a tumor, or infection. Damage to the intervertebral discs and vertebral members can lead to pain, neurological deficit, and/or loss of motion.

Various procedures include replacing the entirety or a section of a vertebral member, the entirety or a section of an intervertebral disc, or both. One or more replacement implants may be inserted to replace the damaged vertebral members and/or discs. The implants are configured to be inserted into the intervertebral space and contact against the remaining adjacent vertebral members.

### SUMMARY

The present application is directed to an implant that fits within a space between skeletal structures and for contacting and spacing apart the skeletal structures. The implant may include a base with first and second sides configured to fit within the space and contact against the skeletal structures. The base may have a height measured between the sides. The implant may include a member having a first height measured between a first pair of opposing sides and a second height measured between a second pair of opposing sides. The first height may be less than or equal to the height of the base and the second height may be greater than the height of the base. The implant may include a connector to connect the member to the base such that the member is able to rotate relative to the base between first and second rotational positions. The member may be rotatable relative to the base between the first rotational position with the first and second sides of the base and the first pair of opposing sides of the member facing in a common direction and one of the second pair of opposing sides facing towards the base, and a second rotational position with the sides of the base and the second pair of opposing sides of the member facing in the common direction and one of the first pair of opposing sides facing towards the base.

The implant may also include a base with a height measured between opposing sides and being sized to fit within the space and contact against the skeletal structures. The implant may include a base a member rotatably connected to the base. The member may also include first and second opposing sides and third and fourth opposing sides. The member may have a first height measured between the first and second sides and a second height measured between the third and fourth sides. The first height may be less than or equal to the height of the base and the second height may be greater than the height of the base. The member may be rotatable relative to the base between a first rotational position with the third side facing towards the intermediate section of the base and a second rotational position with the first side facing towards the intermediate section of the base and the member extending outward beyond the base.

The application also includes a method of spacing apart skeletal structures. The method may include positioning an implant within a space between skeletal structures with the implant including a base and a member rotatably connected to the base. The method may include contacting sides of the base of the implant against the skeletal structures with the member in a first rotational position with at least one of opposing non-contact sides facing towards the skeletal structures and contact sides of the member facing away from the skeletal structures with one of the contact sides facing towards the base. The method may further include that while the sides of the base are contacting the skeletal structures, rotating the member about 90° to a second rotational position and moving the non-contact sides away from the skeletal structures and moving the contact sides of the member into contact with the skeletal structures such that the base and the member simultaneously contact the skeletal structures.

The various aspects of the various embodiments may be used alone or in any combination, as is desired.

### BRIEF DESCRIPTION OF THE DRAWINGS

FIG. 1A is a perspective view of an implant in a first rotational position.

FIG. 1B is a perspective view of the implant of FIG. 1A in a second rotational position.

FIG. 2 is a side schematic view of an implant in a first rotational position positioned in a space between skeletal members.

FIG. 3 is a side schematic view of an implant in a second rotational position positioned in a space between skeletal members.

FIG. 4 is a perspective view of a member having a pair of tabs.

FIG. 5 is a perspective view of a base having an engagement structure.

FIG. 6 is a perspective view of the engagement structure of FIG. 5.

FIG. 7A is a perspective view of an implant in a first rotational position.

FIG. 7B is a perspective view of the implant of FIG. 4A in a second rotational position.

FIG. 8 is a perspective view of an implant in a first rotational position.

FIG. 9 is a sectional view cut along line IX-IX of FIG. 8.

FIG. 10 is a perspective view of the implant of FIG. 8 in a second rotational position.

FIG. 11A is a perspective view of an implant in a first rotational position.

FIG. 11B is a perspective view of the implant of FIG. 5A in a second rotational position.

FIG. 12 is a perspective view of an implant in a second rotational position.

FIG. 13 is a coronal side view of the implant of FIG. 6 in the second rotational position.

FIG. 14 is lateral side view of an implant in a second rotational position.

FIG. 15 is a side view of an implant in a second rotational position.

FIG. 16 is a side view of an implant in a second rotational position.

FIG. 17 is a side view of an implant in a second rotational position.

FIG. 18 is a perspective view of an implant in a first rotational position.

FIG. 19 is a rotational view of the implant of FIG. 18 in a second rotational position.

FIG. 20 is a perspective view of an insertion tool connected to an implant.

FIG. 21 is a sectional view cut along line XXI-XXI of FIG. 20.

FIG. 22 is a perspective view of a distal end of an insertion tool.

FIG. 23 is a perspective view of an implant in a first rotational position.

FIG. 24 is a perspective view of the implant of FIG. 23 in a second rotational position.

### DETAILED DESCRIPTION

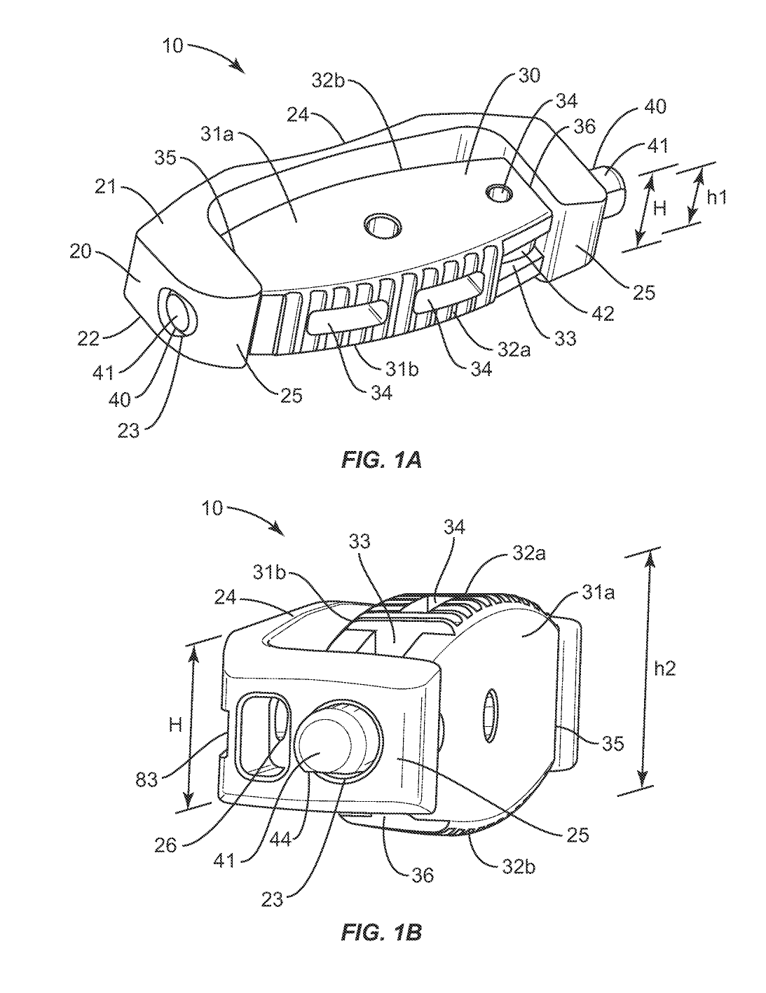

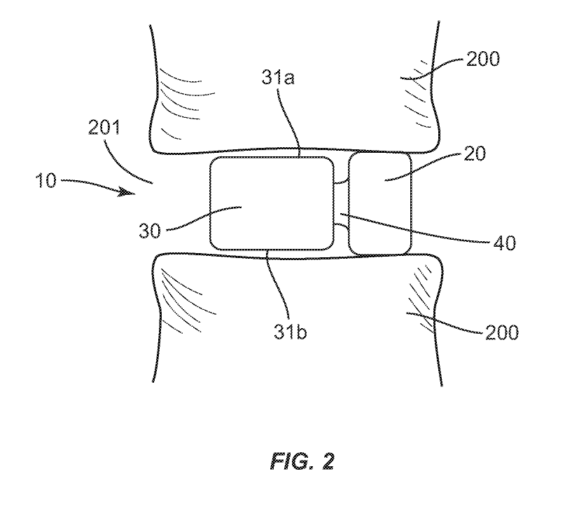

The present application is directed to an implant for spacing apart skeletal structures, such as vertebral members. An implant 10 is illustrated in FIGS. 1A and 1B. The implant 10 includes a base 20, a rotatable member 30, and a connector 40. The base 20 extends around at least a portion of the member 30. The member 30 is movably connected to the base 20 by the connector 40. The member 30 includes a smaller or equal height relative to the base 20 when the member is in a first rotational position as illustrated in FIG. 1A. The member 30 includes a greater height than the base 20 when the member 30 is in a second rotational position as illustrated in FIG. 1B.

FIG. 2 illustrates an implant 10 positioned in a space 201 formed between skeletal members 200. The implant 10 is in a first rotational position that facilitates insertion into the space 201. The base 20 includes a height to contact against each of the skeletal members 200. The member 30 is positioned in the first rotational position with the smaller or equal height. FIG. 2 illustrates the member 30 being spaced from each of the skeletal members 200. The member 30 may also contact both skeletal members 200 or a single one of the skeletal members 200 in this first rotational position.

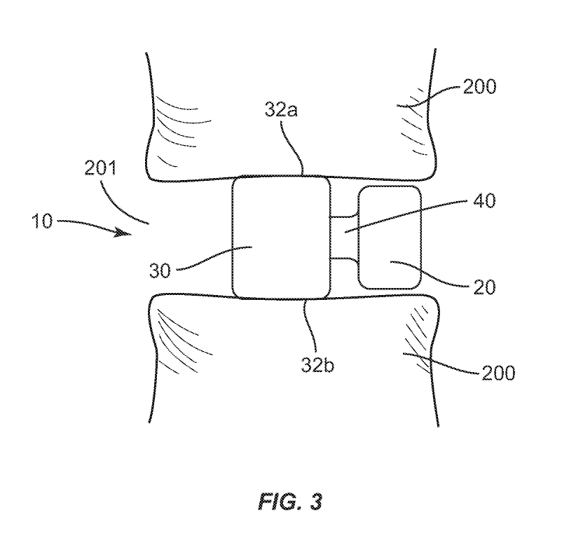

FIG. 3 illustrates the implant 10 in the second rotational position. The member 30 has been moved from the first rotational position to the second rotational position while the implant 10 is within the space 201 and while the base 20 remains in contact with the skeletal members 200. The member 30 in the second rotational position has a larger height and spaces apart the skeletal members 200 a greater amount than in the first rotational position. The shape of the member 30 in the second rotational position complements the base 20 to form substantially continuous overall surfaces for contacting and supporting the skeletal members 200.

Returning to FIGS. 1A and 1B, the base 20 includes a length to extend around at least a portion of the member 30. FIGS. 1A and 1B include the base 20 having an elongated shape with a central section 24 and arms 25 at each of the ends. Base 20 may have various shapes, and may be sized to fit within the space 201 between the skeletal members 200.

The base 20 also includes opposing first and second sides 21, 22 on the central section 24 and arms 25. One or both sides 21, 22 may include contact features such as teeth or surface texturing to facilitate contact with the skeletal members 200 and prevent or reduce movement of the implant 10 after insertion into the space 201. The base 20 includes a height H measured between the sides 21, 22. The height H may be the same along the length of the base 20, or may vary with one or more regions being greater than other regions. FIG. 1B illustrates the height H of the central section 24 being greater than the height of arms 25.

The base 20 may also include one or more openings 23 to receive the one or more connectors 40. The openings 23 may extend completely through the base 20 as illustrated in FIGS. 1A and 1B, or may extend partially into an inner surface of the base 20 that faces towards the member 30. Various other openings 26 may be positioned in the base 26. FIG. 1B includes the arm 25 having an opening 26 in proximity to connector opening 23. This opening 26 is configured to engage with an insertion tool 300 as will be explained in detail below. FIG. 1B includes the opening 26 having a rectangular shape. Opening 26 may also include a circular shape and be threaded.

Additional openings 26 may also extend through the central section 24 for accessing the member 30. In one embodiment, the openings 26 extend through the central section for placing bone growth material between the base 20 and the member 30. Openings 26 may also extend through the height of the base 20 (i.e., through the base 20 from the opposing sides 21, 22). Openings in this orientation may be configured to receive bone growth material.

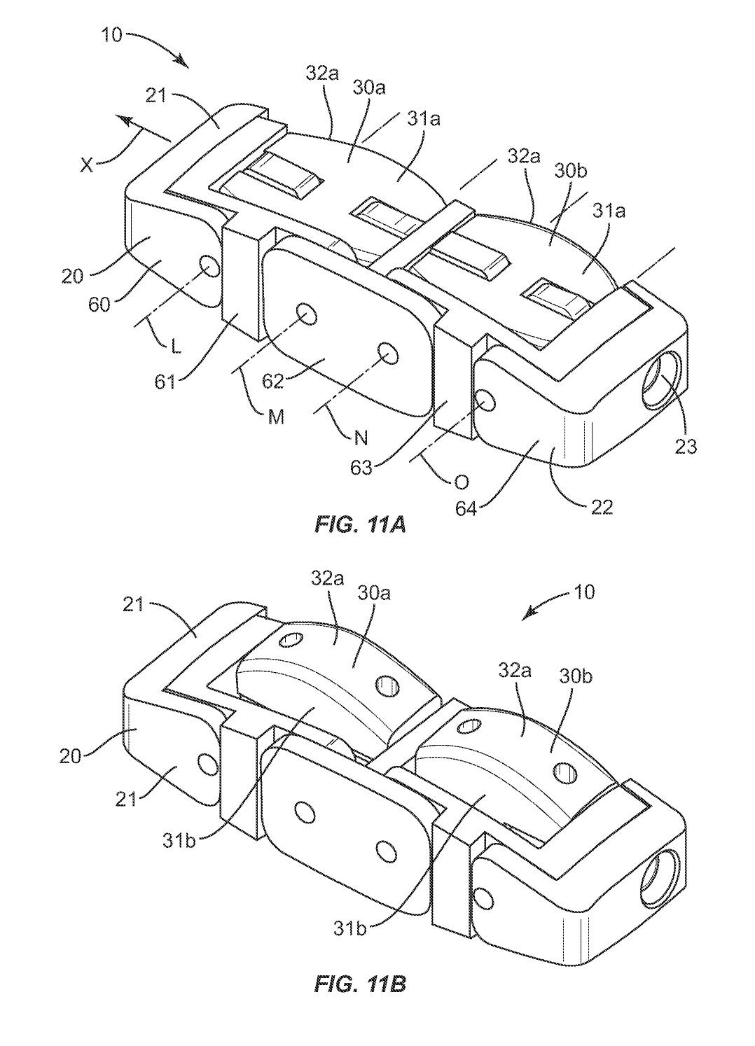

The base 20 may include a single, unitary construction as illustrated in FIGS. 1A and 1B. Other embodiments may include the base 20 constructed of two or more sections that are connected together. FIGS. 11A and 11B include an embodiment with the base 20 constructed from different sections that are pivotally connected together. This provides for the base 20 to articulate during and/or after insertion to facilitate insertion into the space 201 and contact with the skeletal members 200.

The member 30 includes a body with a variety of different sides. FIGS. 1A and 1B include the member 30 having an elongated shape with opposing ends 35, 36. End 35 faces towards one of the arms 25, and the opposing end 36 faces towards the other arm 25. The member 30 also includes sides that extend between the ends 35, 36. A first pair of opposing sides 31a, 31b are spaced apart and include a height h1. These sides 31a, 31b are facing in the same direction as the sides 21, 22 of the base 20 when the member 30 is in the first rotational position as illustrated in FIG. 1A. The height h1 is smaller than or equal to the height H of the base 20. The sides 31a, 31b may not include contact features because the sides face away from the skeletal members 200 when the member 30 is in the second rotational position. The height h1 may be same across the member 30 between the ends 35, 36, or may vary. FIGS. 1A and 1B include the height h1 being substantially the same across the member 30.

Member 30 also includes a second pair of opposing sides 32a, 32b that are spaced apart a greater distance and have a larger height h2. At least one of these sides 32a, 32b faces towards the base 20 when the member 30 is in the first rotational position. FIG. 1A includes that side 32b faces towards the base 20 when the implant 10 is in the first rotational position, and specifically that side 32b faces towards the central section 24 of the base 20. These sides 32a, 32b face in the same direction as the sides 21, 22 of the base 20 when the member 30 is in the second rotational position as illustrated in FIG. 1B. The height h2 is greater than the height H of the base 20 such that the sides 32a, 32b contact against the skeletal members 200. One or both sides 32a, 32b may include contact features such as teeth or surface texturing to facilitate contact with the skeletal members 200. The sides 32a, 32b include a greater amount of contact features than sides 31a, 31b. The height h2 may be the same across the member 30, or may vary. FIGS. 1A and 1B include the height h2 varying across the member 30 with a central section being greater than the ends.

Various openings 34 may extend through the member 30. The openings 34 may extend from side 31a to side 31b, and from sides 32a to side 32b. FIGS. 1A and 1B includes openings 34 extending through the member 30 from sides 32a, 32b. These openings 34 are configured to receive bone growth material and will face towards the skeletal members 200 when the member 30 is in the second rotational position.

The connector 40 pivotally connects the member 30 to the base 20. The connector 40 includes one or more extensions 41 that fit into the corresponding openings 23 in the base 20. The extensions 41 may extend outward from the member 30. The extensions 41 may be part of the member 30 (i.e., an integral, one-piece construction that includes the extensions 41 and the member 30), or the extensions 41 be separate elements that are attached to the member 30.

The extension 41 may include a substantially circular cross-sectional shape with one or more flat sides 44. The one or more flat sides 44 may facilitate engagement with the extension 41. The one or more flat sides 44 may also be a gauge to determine the amount of rotation of the member 30.

One embodiment illustrated in FIG. 1A includes the member 30 with a slot 33 at end 36 that extends through sides 32a and 32b (and between sides 31a and 31b). The connector 40 includes a separate piece connected to the member 30 and including the extension 41 and a body 42. The body 42 is positioned within the slot 33 and connected to the member 30 with a fastener (not illustrated) that extends in the opening 34. The extension 41 extends outward from the end 36 of the member 30 and fits into the opening 23 in the body 20.

In a similar construction, the elements of the base 20 and member 30 are reversed. The base 20 includes the connector 40 that includes one or more extensions 41 that extend outward and fit into corresponding openings in the member 30. These extensions may be integrally constructed to the base 20, or may be separate elements that are attached to the base 20.

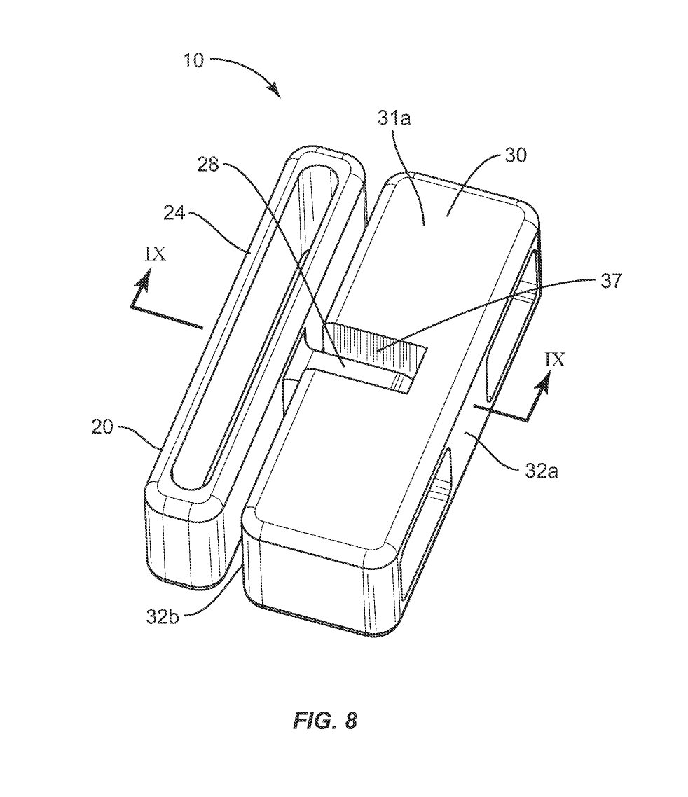

The member 30 may be connected to the base 20 at two or more locations. FIGS. 1A and 1B illustrate and embodiment with two connection locations. Alternatively, the member 30 may be connected to the base 20 at a single location, such as the embodiment illustrated in FIGS. 8, 9, and 10. In the various embodiments with two or more connectors 40, the connectors 40 may be the same or different.

The member 30 may also be connected to the base 20 with a single elongated fastener that extends through the member 30 and into openings 23 in the base 20.

The member 30 rotates about 90° relative to the base 20 between the first and second rotational positions. The connector 40 may be configured to limit an amount of movement between the base 20 and member 30 to within the 90° range. Alternatively, the connector 40 may allow the member 30 to rotate within a greater range (i.e., more than 90°) relative to the body 20.

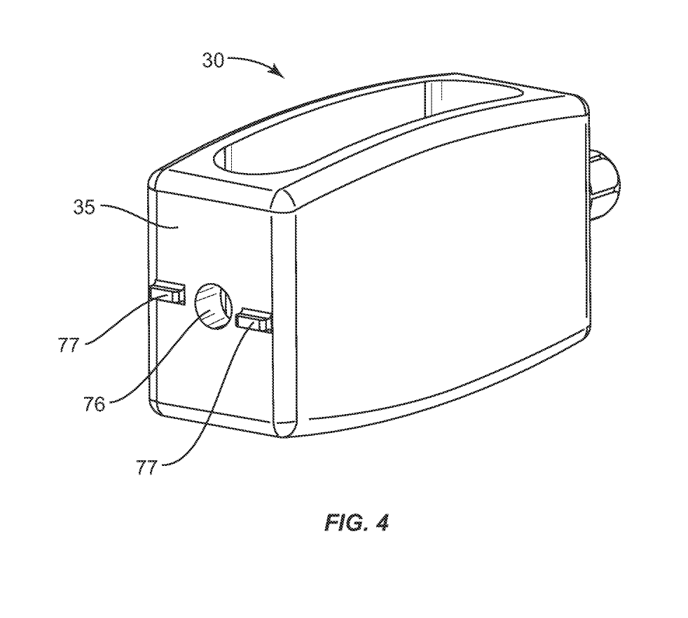

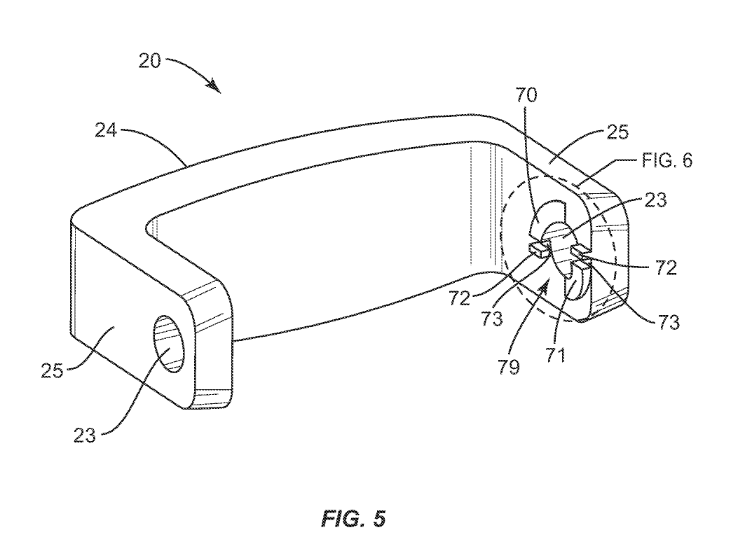

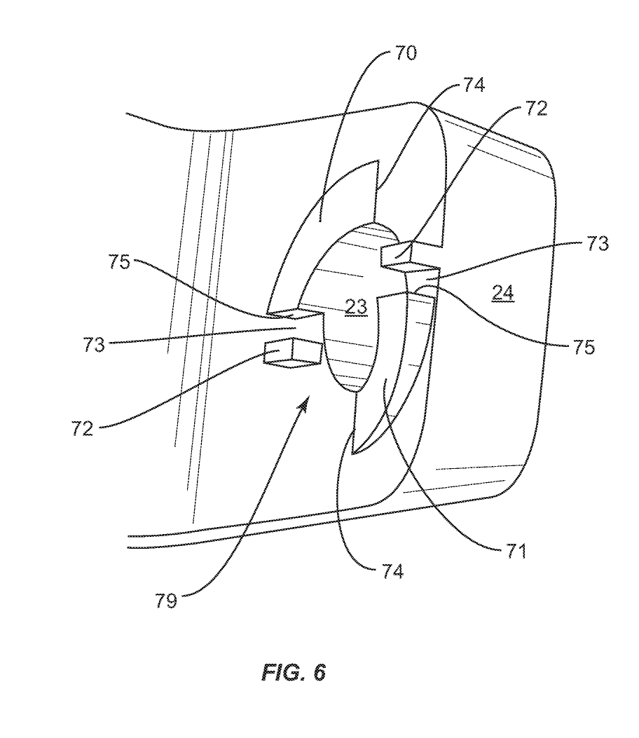

FIGS. 4, 5, and 6 illustrate a structure that limits the amount of rotation between the member 30 and the base 20. The base 20 includes an engagement structure 79 on the inner surface of the arm 25. The engagement structure includes a pair of ramps 70, 71 that extend around sections of the opening 23. Each of the ramps 70, 71 includes a starting point 74 and an ending point 75. The structure in FIGS. 5 and 6 include the ramps 70, 71 increasing in a counter-clockwise direction (as viewed looking at the inner surface of the arm 24). A pair of tabs 72 are positioned in proximity to the ending points 75 and form slots 73 adjacent to the ending points 75. As illustrated in FIG. 4, the member 30 includes an end 35 with a pair of tabs 77 positioned on opposing sides of an opening 76.

The member 30 is attached to the base 20 with a fastener (not illustrated) that extends through and aligns the openings 23, 76. As the member 30 is rotated from the first rotational position to the second rotational position, the tabs 77 on the member 30 slide along the respective ramps 70, 71 from the starting points 74 towards the ending points 75. The member 30 is rotated and the tabs 77 slide beyond the end points 75 and into the slots 73. The tabs 77 are captured in the slots 73 thus securing the member 30 in the second rotational position.

The implant 10 may include a single engagement structure 79 and corresponding slots 73, or may include multiple structures 79. FIG. 4 includes a pair of tabs 77 extending from the member 30. The member 30 may also include a single tab 77, or three or more tabs 77. Further, FIGS. 4, 5, and 6 include the engagement structure 79 on the base 20 and the tabs 77 on the member 30. These elements may be reversed with the engagement structure 79 on the member 30 and the tabs 77 on the base 20.

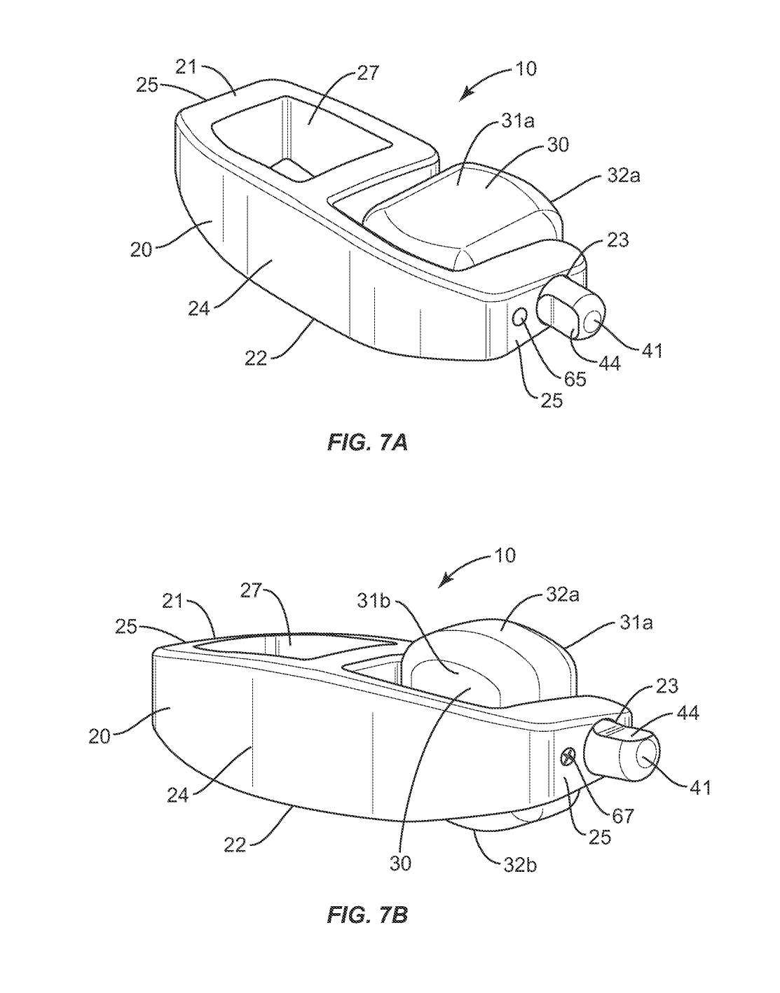

The relative sizes between the body 20 and the member 30 may vary. FIGS. 1A and 1B include the body 20 with a slightly greater length than the member 30. The central section 24 is about the same length as the member 30, with the arms 25 extending outward beyond the member 30. The body 20 may also include a considerably greater length than the member 30. FIGS. 7A and 7B include an implant 10 with the body 20 having a section with an opening 27 positioned outward beyond the length of the member 30. The opening 27 extends through the first and second sides 21, 22 of the base 20 and may hold bone growth material to facilitate fusion between the skeletal members 200. Other embodiments may include the length of the member 30 may being equal to or greater than the length of the body 20 (e.g., FIGS. 8, 9, and 10).

The member 30 may be connected to the base 20 at opposing ends as illustrated in FIGS. 1A and 1B. Member 30 may also be connected in other manners. FIGS. 8, 9, and 10 include the member 30 attached at a single location. Base 20 includes an extension 28 that extends outward from one side of the central section 24. The member 30 includes a slot 37 sized to receive the extension 28. The slot 37 extends inward from one of the sides 31a, 31b, and one of sides 32a, 32b. A fastener 60 connects the extension 28 to the member 30 and allows for rotation of the member 30 relative to the base 20.

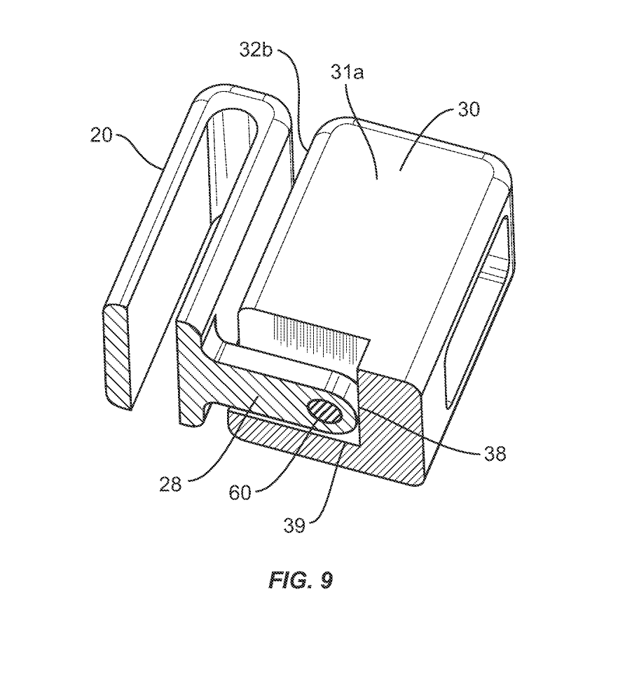

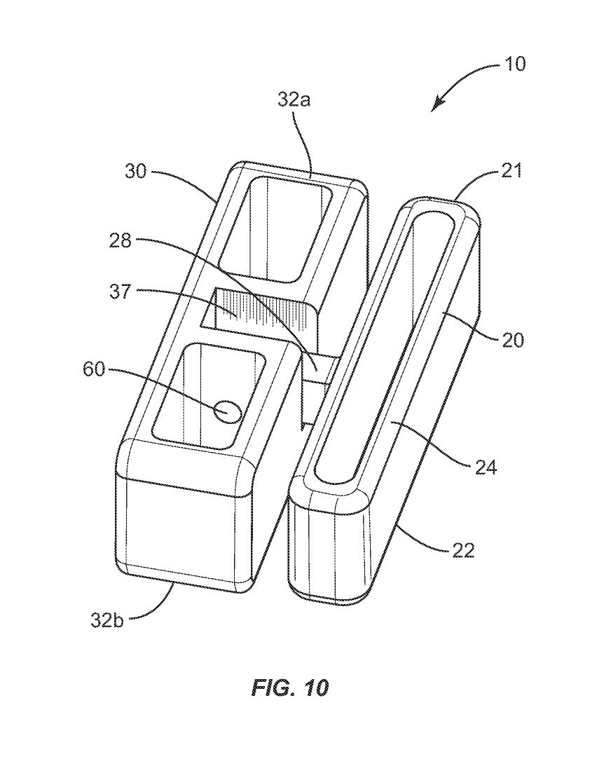

FIGS. 8 and 9 illustrate the implant 10 in the first rotational position. The extension 28 abuts against side 39 of the slot 37 to limit the extent of rotation in a first direction. FIG. 10 illustrates the member 30 rotated about 90° to the second rotational position. The opposing side of the extension 28 abuts against the other side 38 of the slot 37 to limit the extent of rotation in the second direction. The amount of angular rotation between the first and second rotational positions is controlled by the relative angular positions of the sides 38, 39. The amount of angular rotation may vary.

FIGS. 8 and 10 include the lengths of the base 20 and member 30 being substantially the same. The lengths may also be different with the base 20 being longer, equal to, or shorter than the member 30.

Two or more members 30 may be included in an implant 10. FIGS. 11A and 11B include a pair of members 30a, 30b positioned within the body 20. As with the other embodiment, each of the members 30a, 30b includes a first pair of sides 31a, 31b, and a second pair of sides 32a, 32b. The members 30 are each movable between first and second rotational positions. In embodiments with multiple members 30, the members 30 may include the same shape and size (as illustrated in FIGS. 11A and 11B), or may include different shapes and/or sizes.

In embodiments with multiple members 30, the members 30 may be independently rotatable, or may rotate together. In one embodiment, the multiple members 30 are connected together. A rotational force applied to one of the members 30 results in each of the members 30 being rotated between first and second rotational positions. In other embodiments, the members 30 remain independent and each must be individually rotated between the first and second rotational positions. In embodiments with three or more members 30, subsets of the members 30 may be connected to rotate together (e.g., two members are connected with a third being independent).

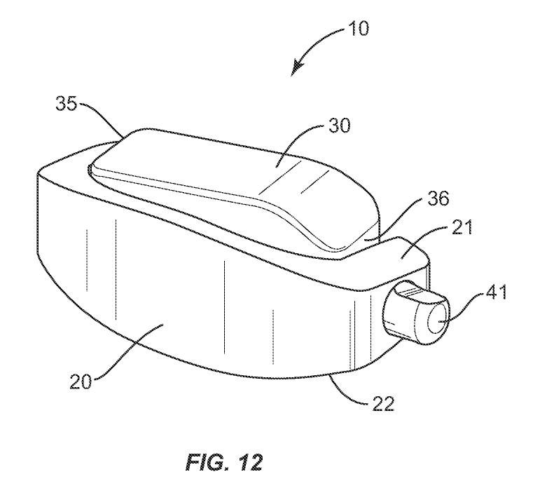

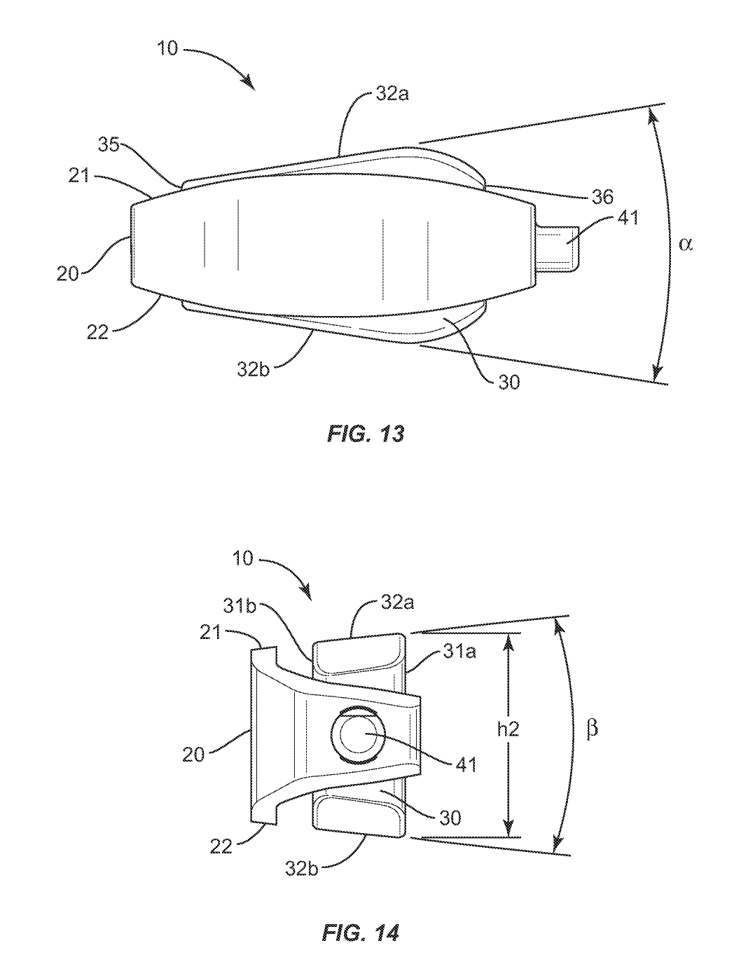

The shape of the members 30 may vary. FIGS. 1A and 1B include a member 30 having substantially rounded sides 32a, 32b with the height h2 greatest at a central portion and tapering to smaller heights at each of the opposing ends 35, 36. FIG. 12 includes a member 30 with reduced height at end 35 that increases to a maximum height in a central area in proximity to the opposing end 36. As illustrated in FIG. 13, this shape includes a correction angle α formed by opposing sides of the implant 10. In one embodiment, the correction angle α is aligned in the coronal plane. The correction angle α may range from between about 0° to about 30°. In some specific applications of the implant 10 positioned in the coronal plane, the angle α may be as high as 45°.

The members 30 may have various heights across the contact sides 32a, 32b. FIG. 1B illustrates the height h2 between the sides 32a, 32b being substantially constant. FIG. 14 includes a member 30 with the height h2 varying across the sides 32a, 32b. The height h2 is greatest at an intersection with side 31a and gradually decreases across the sides 32a, 32b to a minimum at an intersection with side 31b. As illustrated in FIG. 14, a correction angle β is formed between the opposing sides. The correction angle f3 may range from between about 0° to about 30°.

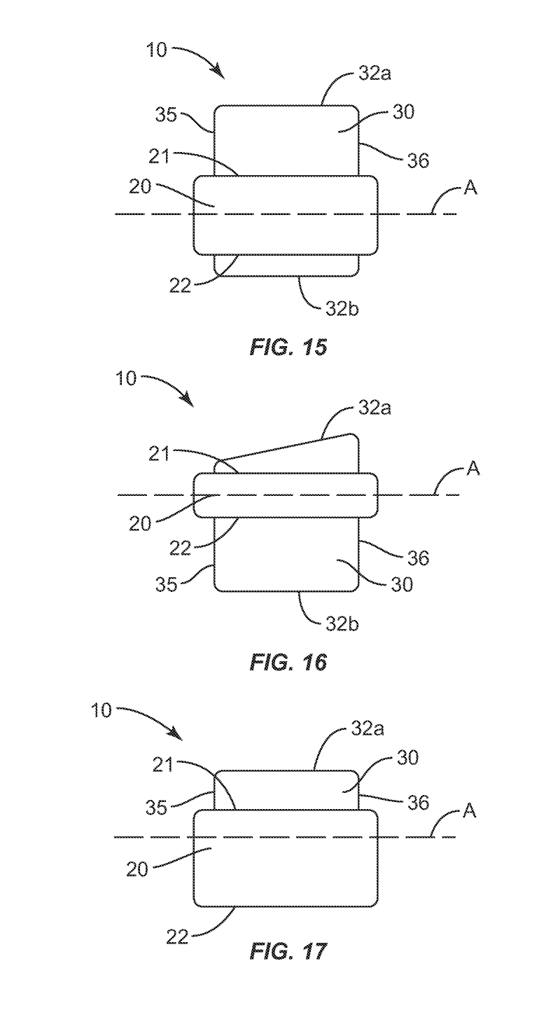

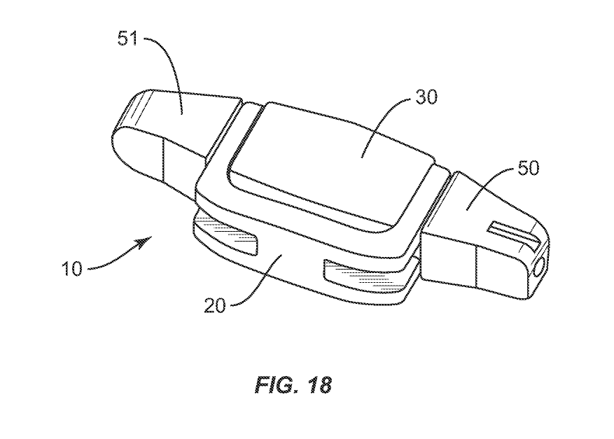

The member 30 may extend outward from the base 20 in different manners when the member 30 is in the second rotational position. FIGS. 1B, 13, and 14 each include the member 30 extending outward an equal amount beyond each of the sides 21, 22. Member 30 may also be configured to extend outward from the sides 21, 22 different amounts. FIG. 15 includes an embodiment in the second rotational position with the member 30 extending outward beyond the first side 21 a greater amount that from the second side 22. FIG. 16 includes the member 30 extending outward a greater beyond side 22 than beyond side 21. FIG. 17 includes the member 30 extending outward only beyond side 21. The opposing side 32b (not illustrated in FIG. 17) may be positioned inward from side 22, or may be flush with side 22. For ease of reference, a rotational axis A is illustrated in each of FIGS. 15-17.

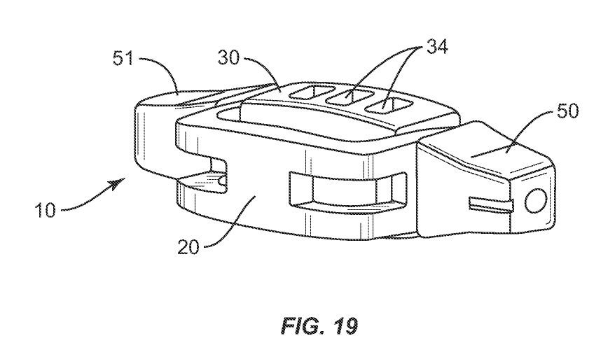

The implant 10 may also include one or more wings 50, 51 operatively connected to the member 30 as illustrated in FIGS. 18 and 19. The wings 50, 51 extend outward on opposing sides of the member 30 to further support the skeletal members 200. The wings 50, 51 may have a smaller height, equal height, or greater height relative to the base 20 and member 30 in either the first rotational position (FIG. 18) or the second rotational position (FIG. 19). The shape of the wings 50, 51 in the second rotational position may complement the base 20 and/or member 30 to provide a continuous shape for supporting the skeletal members 200. The wings 50, 51 may each include the same shape and size and illustrated in FIGS. 18 and 19, or may include different shapes and/or sizes. FIGS. 18 and 19 include two wings with the first wing 50 operatively connected to the first side of the implant 10, and the second wing 50 operatively connected to the second side. Implant 10 may also include a single wing attached to one of the first and second sides.

Each of the wings 50, 51 may be connected to one of the extensions 41 that extend outward from the base 20. Alternatively, one or more fasteners may extend outward from the member 30 and connect to the wings 50, 51.

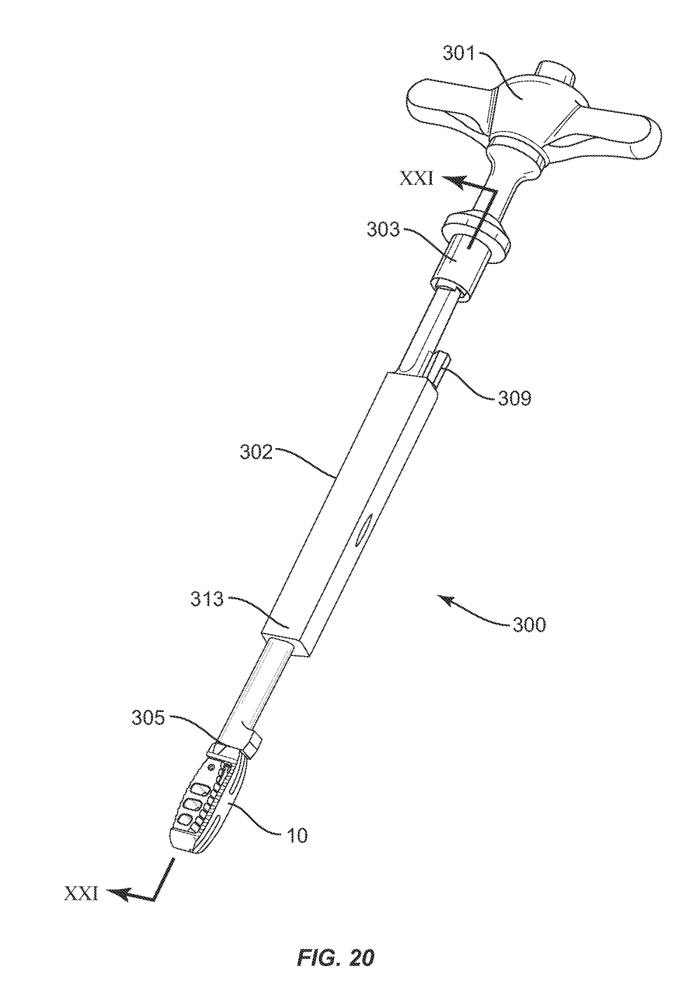

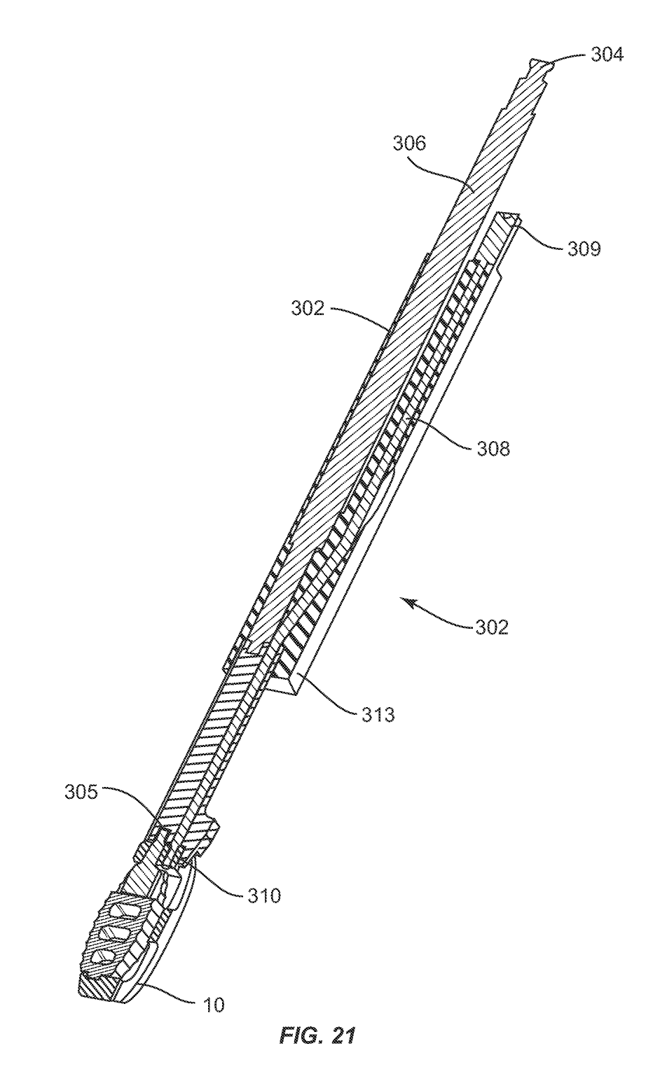

The implant 10 may be inserted into the space 201 with an insertion tool 300 as illustrated in FIGS. 20, 21, and 22. The insertion tool 300 generally includes a handle 301 and an elongated section 302. The handle 301 includes a connector 303 and may be permanently connected to the elongated section 302, or may be removably connected to the section 302.

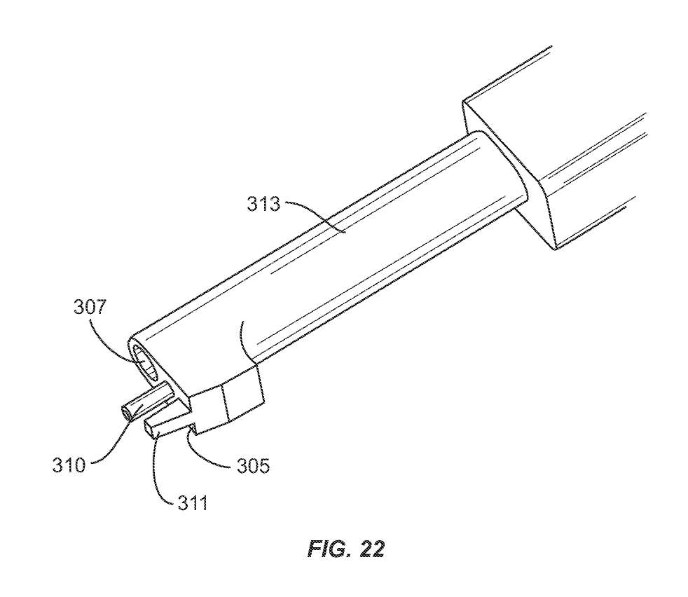

The elongated section 302 includes a housing 313 that extends around all or a portion of a shaft 306 and a driver 308. The housing 313 is hollow to extend around the shaft 306 and the driver 308. As illustrated in FIG. 22, an extension 311 extends outward from the distal end of the housing 313 to engage with the slot 83 in the implant 10.

The shaft 306 is rotatable within the housing 313 and includes a proximal end 304 and an opposing distal end 305. The proximal end 304 is positioned outward beyond a proximal end of the housing 313 and is configured to connect to the connector 303. The distal end 305 of the shaft 306 may include a receptacle 307 sized to engage with the extension 41 on the implant 10. The receptacle 307 may include one or more flat sides that align with and engage the flat sides 44 of the extension 41. Rotation of the handle 301 causes the shaft 302 to rotate thus moving the member 30 from the first rotational position to the second rotational position. The shaft 306 may be constructed from a single continuous piece, or may include two or more separate pieces that are connected together.

The driver 308 extends through the housing 313 and is configured to also engage with the implant 10. The driver 308 includes a proximal end with a knob 309 that is positioned on the exterior of the housing 313, and a distal end that forms an extension 310 that extends outward from the distal end of the housing 313. The extension 310 may be threaded to engage within a threaded opening 26 in the implant 10. The driver 308 is also rotatable relative to the housing 313 such that rotation of the knob 309 rotates the driver 308 causing extension 310 to thread into the opening 26. The driver 308 may be flexible as it meanders through the interior of the housing 313.

In use, the implant 10 is initially connected to the insertion tool 300. This includes placing the implant 10 at the distal end 305 of the elongated section 302. The extension 311 on the housing 313 is positioned in the slot 83 in the implant 10. Further, the extension 310 on the driver 308 is inserted into the opening 26 in the implant 10. This may include surgical personnel rotating the knob 309 on the proximal end of the driver 308 and threading the extension 310 into the threaded opening 26 in the implant 10. Further, the receptacle 307 at the distal end of the shaft 306 is placed over and engages the extension 41 on the implant 10.

The implant 10 is positioned in the first rotational position. This provides a reduced overall height and facilitates insertion into the patient and into the space 201 between the skeletal members 200. The surgical personnel manipulate the insertion tool 300 and the implant 10 is placed in the space 201 with the side 21 of the base 20 contacting against the first skeletal member 200 and side 22 contacting against the second skeletal member 200. With the member 30 in the first rotational position, the sides 31a, 31b are facing in the same direction as sides 21, 22 and towards the skeletal members 200. One, both, or neither of the sides 31a, 31b may be in contact with the skeletal members 200.

Bone growth material may be placed into the implant 10 prior to or after the implant 10 is positioned in the space 201. After the implant 10 is properly positioned in the space 201, the member 30 is rotated to the second rotational position. This includes rotating the handle 301 on the insertion tool 300 thereby rotating the shaft 306 and hence the member 30.

In some embodiments, the extent of rotation of the member 30 is performed visually by the surgical personnel. They may observe the flat side 44 of the extension and determine when the member 30 has been rotated the correct amount. The surgical personnel may also use tactile feedback to determine rotation. Other embodiments may include limiters to control the extent of rotation. This may include engagement structures 79 as illustrated in FIGS. 4, 5, and 6. In one embodiment, the extension 41 includes an outwardly-extending flange that contacts against a shelf on the base 20 to limit an extent of rotation at the second rotational position. A similar construction may also be formed to control the extent of rotation at the first rotational position. Various other mechanisms may be employed to limit an extent of rotation of the member 30. The insertion tool 300 may further include a gauge that indicates the amount of rotation of the member 30.

Rotation of the member 30 to the second rotational position causes the sides 32a, 32b to move into contact with the skeletal members 200. The height h2 of the member 30 is configured to support the skeletal members 200 at the proper spacing.

The member 30 may be secured after rotation to the second rotational position. A fastener 67 (see FIG. 7B) may be inserted into an opening 65 in the base 20 and into contact with the member 30 to maintain the rotational position. Another embodiment includes a fastener placed into contact with the extension 41 to maintain the rotational position.

After the implant 10 is properly positioned in the space 201 while in the second rotational position, the insertion tool 300 may be removed from the implant 10. This may include rotating the knob 309 and removing the extension 310 from the implant 10. Further, the insertion tool 300 is moved outward away from the implant 10 to disengage from the implant 10.

The implant 10 may also be manually inserted into the space 201 and rotated to the second rotational position.

The base 20 may be constructed to have an articulating configuration. Base 20 includes multiple sections that are connected together and provide for pivoting motion between the sections. FIGS. 11A and 11B include an embodiment with the base 20 constructed from different sections 60, 61, 62, 63, and 64. These different sections are pivotally connected along pivot axes L, M, N, and O. This causes the implant 10 to assume different shapes during insertion into the space 201, and after the implant 10 has been inserted into the space 201 to facilitate contact with the skeletal members 200. By way of example, as the implant 10 is being inserted in the direction of arrow X, the base 20 will articulate along one or more of the axes L, M, N, O. The different sections 60, 61, 62, 63, 64 may also be positioned at different angular positions after the implant 10 is within the space 201. The articulating structure of the base 20 still provides for one or more members 30 to be connected and to be movable between the first and second rotational positions. Various examples of articulating structures are disclosed in U.S. patent application Ser. Nos. 12/971,861 entitled “Flexible Spinal Implant” and filed on Jan. 4, 2011, 12/533,877 and 12/605,415, each of which are assigned to the same entity as the present application and each of which is herein incorporated by reference in its entirety.

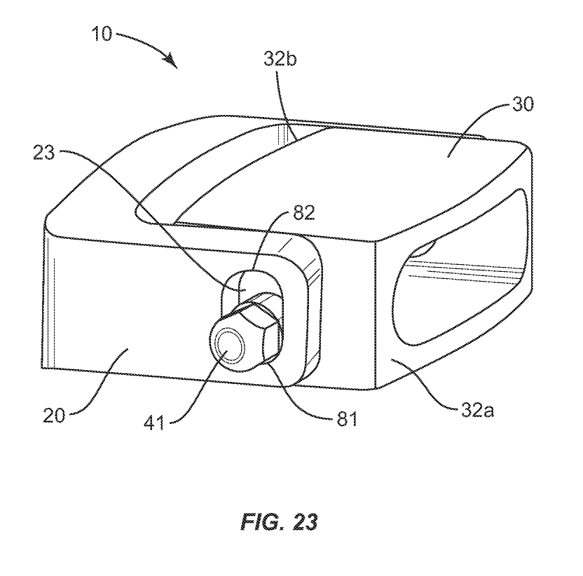

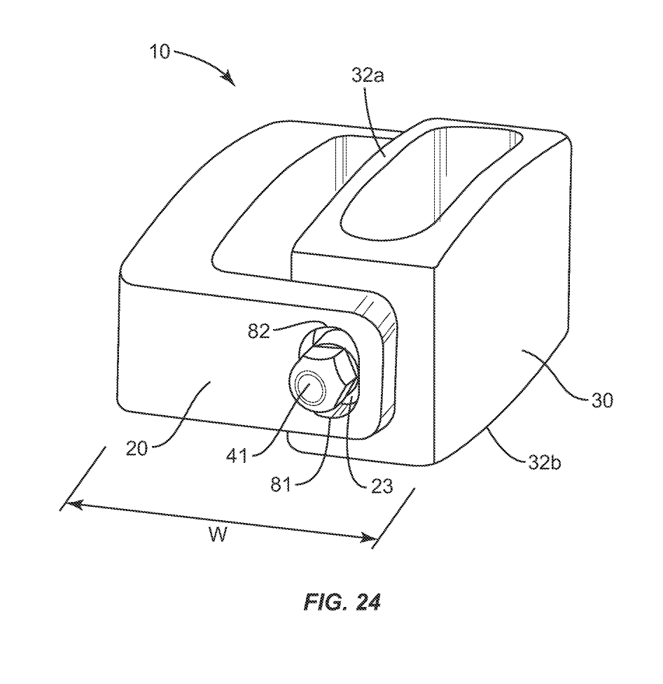

FIGS. 23 and 24 illustrate that the size of the opening 23 in the base 20 may be larger than the size of the extension 41. The illustrated embodiment includes the opening 23 having an elongated shape with a larger height defined between ends 81, 82, and smaller width. Further, the extension 41 is offset from a center of the member 30 between sides 31a and 31b (i.e., the extension 41 is closer to one of the sides 31a, 31b than the other side).

During rotation of the member 30, the extension 41 rotates and translates in the opening 23. FIG. 23 illustrates the implant 10 in the first rotational position with the extension 41 positioned towards the end 81 of the opening 23. FIG. 24 illustrates the implant 10 in the second rotation position with the extension 41 having translated towards the center of the opening 23 (i.e., away from end 81). This construction shifts the member 30 away from the midline of the base 20 as it is rotated to keep the side-to-side width W of the implant 10 from decreasing too much during rotation of the member 30. FIG. 24 includes the width W extending being formed in part by the base 20 and in part by the member 30.

In some embodiments, the rotational axis A is centered in the member 30. Other embodiments may include the rotational axis A is offset in the member 30. This may include offset between sides 32a and 32b, and/or between sides 31a, 31b.

A rotatable implant is also disclosed in U.S. patent application Ser. No. 12/845,809 filed Jul. 30, 2010 which is herein incorporated by reference in its entirety.

In one embodiment, the implant 10 includes a base 20 with a height of 10 mm for insertion when the member 30 is in the first rotational position, and includes the member 30 with a height of 14 mm for supporting the skeletal members 200 when the member 30 is in the second rotational position.

The height h1 of the member 30 in the first rotational position may be smaller than the height H of the base 20. The height h1 may also be equal to the height H.

The implant 10 is sized to space apart skeletal members 200. One application includes the implant 10 positioned within the intervertebral space between the bodies of vertebral members. The implant 10 may also be used to treat long bones (e.g., femur, tibia, fibula, humerus).

The various elements of the implant 10 may be constructed of biocompatible materials of various types. Examples of implant materials include, but are not limited to, non-reinforced polymers, reinforced polymer composites, PEEK and PEEK composites, shape-memory alloys, titanium, titanium alloys, cobalt chrome alloys, stainless steel, ceramics and combinations thereof. Reinforcing materials may include carbon, fiberglass, metal pieces, or any other effective reinforcing material. The implant 10 may include radiographic markers to provide the ability to monitor and determine radiographically or fluoroscopically the location of the implant 10 within the patient. In some embodiments, the implant elements may be constructed of solid sections of bone or other tissues. In other embodiments, the implant elements are constructed of planks of bone that are assembled into a final configuration. The implant may be constructed of planks of bone that are assembled along horizontal or vertical planes through one or more longitudinal axes of the implant. Tissue materials include, but are not limited to, synthetic or natural autograft, allograft or xenograft, and may be resorbable or non-resorbable in nature. Examples of other tissue materials include, but are not limited to, hard tissues, connective tissues, demineralized bone matrix and combinations thereof. Examples of resorbable materials that may be used include, but are not limited to, polylactide, polyglycolide, tyrosine-derived polycarbonate, polyanhydride, polyorthoester, polyphosphazene, calcium phosphate, hydroxyapatite, bioactive glass, and combinations thereof.

The implant 10 may be used during surgical procedures on living patients. The implant 10 may also be used in a non-living situation, such as within a cadaver, model, and the like. The non-living situation may be for one or more of testing, training, and demonstration purposes.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. An implant to fit within a space between skeletal structures and for contacting and spacing apart the skeletal structures, the implant comprising:
a base with first and second sides configured to fit within the space and contact against the skeletal structures, the base having a height measured between the sides;
a member having a first height measured between a first pair of opposing sides and a second height measured between a second pair of opposing sides, the first height being less than or equal to the height of the base and the second height being greater than the height of the base;
a connector to connect the member to the base such that the member is able to rotate relative to the base between first and second rotational positions;
the member rotatable relative to the base between the first rotational position with the first and second sides of the base and the first pair of opposing sides of the member facing in a common direction and one of the second pair of opposing sides facing towards the base, and a second rotational position with the sides of the base and the second pair of opposing sides of the member facing in the common direction and one of the first pair of opposing sides facing towards the base.

2. The implant of claim 1, wherein the connector includes a first connection section that extends between the base and the member and connects a first end of the member to the base, and a second connection section that extends between the base and the member and connects a second end of the member to the base, the first and second connection sections configured for the member to rotate about 90° between the first and second rotational positions.

3. The implant of claim 2, wherein each of the first and second connection sections includes an extension that extends outward from the member and fits into an opening in the base.

4. The implant of claim 1, wherein the second pair of opposing sides extends outward beyond both the first and second sides of the base in the second rotational position.

5. The implant of claim 1, wherein the base includes first and second arms with an intermediate central section that extends between the arms, the member being connected to the base at each of the first and second arms and the one of the second pair of opposing sides facing towards the central section when the member is in the first rotational position.

6. The implant of claim 1, further comprising a wing operatively connected to the member and rotatable with the member, the wing positioned on an opposing side of the base from the member.

7. The implant of claim 1, wherein the second height of the member varies across the member.

8. The implant of claim 1, wherein the base is constructed from multiple sections that are connected together in an articulating manner.

9. An implant to fit within a space between skeletal structures and for contacting and spacing apart the skeletal structures, the implant comprising:
a base with a height measured between opposing sides and being sized to fit within the space and contact against the skeletal structures, the base including an intermediate section positioned between first and second sections;
a member connected to the base and including first and second opposing sides and third and fourth opposing sides, the member having a first height measured between the first and second sides and a second height measured between the third and fourth sides, the first height being less than or equal to the height of the base and the second height being greater than the height of the base;
the member rotatable relative to the base between a first rotational position with the third side facing towards the intermediate section of the base and a second rotational position with the first side facing towards the intermediate section of the base and the member extending outward beyond at least one of the opposing sides of the base.

10. The implant of claim 9, wherein the base extends around the member.

11. The implant of claim 9, further comprising a second member connected to the base and being rotatable relative to the base.

12. The implant of claim 9, wherein the second member is operatively connected to the member to rotate together.

13. The implant of claim 9, wherein one of the base and the member includes a ramp with a starting point and an end point and the other of the base and the member includes a tab, the ramp and the tab contacting each other during rotation of the member from the first rotational position to the second rotational position.

14. The implant of claim 9, further comprising extensions that extend outward from member and fit respectively within first and second openings in the base.

15. The implant of claim 9, wherein the first and second sides of the member face in the same direction as the sides of the base at the first rotational position, and the third and fourth sides of the member face in the same direction as the sides of the base at the second rotational position, with the first and second rotational positions being about 90° apart.

16. A method of spacing apart skeletal structures comprising:
positioning an implant within a space between skeletal structures, the implant including a base and a member rotatably connected to the base and contacting sides of the base of the implant against the skeletal structures with the member in a first rotational position with at least one of opposing non-contact sides facing towards the skeletal structures and contact sides of the member facing away from the skeletal structures with one of the contact sides facing towards the base, the non-contact sides of the member being positioned flush with or below the contacting sides of the base; and
while the sides of the base are contacting the skeletal structures, rotating the member about 90° to a second rotational position and moving the non-contact sides away from the skeletal structures and moving the contact sides of the member into contact with the skeletal structures such that the base and the member simultaneously contact the skeletal structures, the contact sides of the member positioned outward from the contacting sides of the base.

17. The method of claim 16, further comprising rotating a tab on one of the base and the member along a ramped surface on the other of the base and the member.

18. The method of claim 16, further comprising rotating extensions that extend outward from one of the member and the base within openings in the other of the member and the base.

19. The method of claim 16, wherein rotating the member further comprises rotating a second member rotatably connected to the base.

20. The method of claim 16, further comprising articulating sections of the base while positioning the implant within the space between the skeletal structures.
